(19) United States
(12) Patent Application Publication
(10) Pub. No.: US 2005/0222264 A1
(43) Pub. Date: Oct. 6, 2005

(54) Anti-rhinovirus active agents

(75) Inventor: Giulio Tarro, Napoli (IT)

Correspondence Address: YOUNG & THOMPSON, 745 SOUTH 23RD STREET, 2ND FLOOR, ARLINGTON, VA, 22202, US

(73) Assignee: UNIHART CORPORATION, DUBLIN 2 (IE)

(21) Appl. No.: 10/513,580

(22) PCT Filed: May 5, 2003
(86) PCT No.: PCT/EP03/04673

§ 371 Date: Nov. 5, 2004

(30) Foreign Application Priority Data
May 8, 2002 (IT) MI2002A000966

(51) Int. Cl. A61K 31/195; A61K 31/12

(52) U.S. Cl. 514/567; 514/679

(57) ABSTRACT

Glyoxal derivatives for use in the prevention and treatment of Rhinovirus infections are herein disclosed.

The present invention relates to the use of glyoxal derivatives for the treatment and the prevention of Rhinovirus infections.

Human Rhinoviruses (HRV) belong to the family of piconaviruses and are regarded as the causal agents of the common cold.

Rhinoviruses infect the host cells through binding to specific receptors, mainly protein ICAM-1, which belongs to the immunoglobulin family and functions as a ligand for LFA-1 (lymphocyte function associated molecule-1).

Among the different approaches hitherto made to reduce the infectivity of Rhinoviruses, we can mention the following:

1) development of anti-surface-receptor antibodies that block virus binding, 2) use of interferon to induce an antiviral response, 3) development of viral replication inhibitors and 4) development of antibodies against the proteins of the viral capside. Moreover, said approaches revealed often unsatisfactory, due to the onset of undesired side effects. For example, it has been observed that nasal administration of interferon, although reducing the severity of the viral infection, causes bleeding of the nasal mucosa.

Glyoxal derivatives are a class of structurally homogeneous compounds characterized by the presence of a keto-aldehydic function. Among them, biphenyl keto-aldehydic derivatives, in particular xenalamine and xenaldial, are known to possess antiviral activity against influenza virus (A-PR8), murine hepatitis virus (MHV-3) and graig, aphta, measles and polio-1 viruses (see biblography).

It has now been found that biphenyl-, naphthyl- and cumaroyl-glyoxal compounds are able to stop the infection triggered by different strains of Rhinoviruses by preventing the virus from penetrating in the cell.

Object of the invention is therefore the use of a compound selected from the group consisting of:
1) p-(α-ethoxy-p-phenyl-phenacylamino)-benzoic acid (xenalamine): 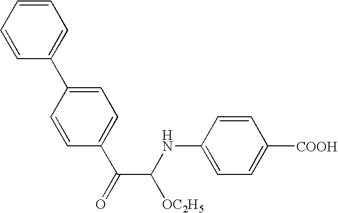
2) 4,4′-bis-biphenylglyoxal-hydrate (xenaldial): 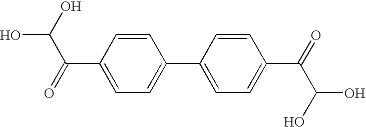
3) p-(α-ethoxy-β-keto-β-1-naphthyl)-ethylamino-benzoic acid (DV1): 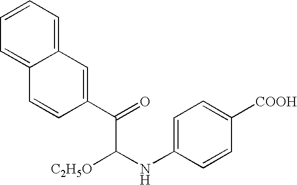
4) p-(α-ethoxy-β-keto-β-2-naphthyl)-ethylamino-benzoic acid (DV2): 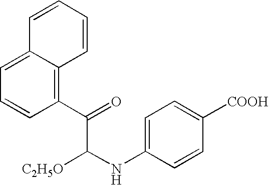
5) p-(α-ethoxy-β-keto-β-2-cumaroyl)-ethylamino-benzoic acid (DV3): 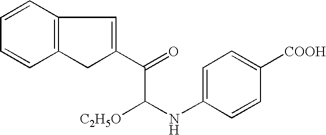
6) p-(α-ethoxy-β-keto-β-1-phenanthryl)-ethylamino-benzoic acid (DV4): 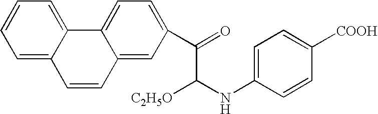
7) p-(α-ethoxy-β-keto-β-2-phenanthryl)-ethylamino-benzoic acid (DV5): 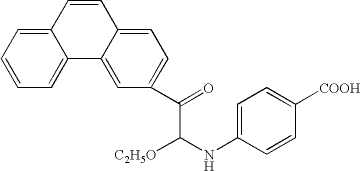
   for the preparation of a medicament for use in the prevention or treatment of Rhinovirus infections, in particular of colds.

The activity of the above compounds was evaluated in a plaque inhibition assay carried out on mono-layers of kidney cells from Rhesus monkey and from WI38 and Ohio-HeLa human cells, suitable for the propagation of Rhinoviruses M and H strains respectively. In this assay, the addition to the culture medium of an agent able to impede the viral penetration in the cells reduces the formation of microplaques compared with an untreated control culture. Therefore, the effectiveness of the test compound can be determined by counting the microplaques in treated and non-treated cultures after exposure to the virus.

The results show that the test compounds are able to prevent or stop the infectious process caused by Rhinoviruses. Moreover, the compounds are devoid of toxicity towards the treated cells. Although the observed effects are probably due to receptor block, the invention is not in any way bound to a specific mechanism of action.

According to a further aspect, the invention relates to pharmaceutical compositions containing at least one of the above mentioned compounds in combination with pharmaceutically acceptable carriers and excipients. According to a preferred embodiment, the compositions are in a form suitable for inhalatory administration. Preferred pharmaceutical forms are solutions, suspensions, dispersions, powders and granulates for the oral or nasal administration by means of spray and aerosol. These can be prepared with conventional techniques, for example as described in Remington's Pharmaceutical Sciences Handbook, Mack Pub. Co., Ny, USA, XVII Ed.

The effective dose of active ingredient can range from 0.1 to 10 mg/Kg/die, preferably 1 mg/Kg/die.

The invention is illustrated by means of the following examples.

### EXAMPLE 1

### Plaques Assay on Cell Mono-Layers

Procedure:

1. prepare confluent kidney cell mono-layers from Rhesus monkeys and from human cell lines (WI 38, MRC-5 and Ohio-Hela cells) in 6.75 cm Ø Petri dishes;
2. aspirate the growth medium;
3. wash each dish with 10 ml of culture medium (serum-free growth medium), Earle's saline solution—lactoalbumine hydrolysate and incubate at 33° C. for 60′;
4. aspirate the liquid;
5. prepare serial 1:10 viral dilutions (Rhinovirus-strain M and Rhinovirus-strain H) in Earle's saline solution—lactoalbumine hydrolysate and inoculate each dish with 0.2 ml volumes of viral dilution. Each viral dilution is inoculated in duplicate.
6. Prepare analogous serial viral dilutions (as in 5) in Earle's saline solution—lactoalbumine hydrolysate, added with the anti-viral test compound at the desired concentration.
7. Prepare Earle's saline solution—lactoalbumine hydrolysate added with the test anti-viral compound at the desired concentration as the control (one control for each test compound).
8. incubate the infected mono-layers at 33° C. for 60′, gently rocking the dishes at 15′ intervals in order to evenly distribute the inoculum;
9. place the dishes on a flat surface;
10. thoroughly add 10 ml of nutritive agar in Earle's saline solution—calf serum, equilibrated at 44° C., to each dish and allow to harden. This step must be carried out under attenuated light;
11. close the dishes with the lid and incubate in reversed position at 33° C., in the dark, under 5% CO₂ humidified atmosphere;
12. count the microplaques after 3 days incubation: they will appear as pale, approximately circular areas against a pink background.

1) Virus: Rhinovirus H Strain, Cell Line: WI38

Results:

| | | | DV1 | DV2 | DV3 | DV4 | DV5 | |
|---|---|---|---|---|---|---|---|---|
| Dilution | Non-treated | Xenaldial | n^{o} of | n^{o} of | n^{o} of | n^{o} of | n^{o} of | Xenalamine |
| of | control n^{o} | n^{o} of | plaques | plaques | plaques | plaques | plaques | n^{o} of |
| inoculated | of plaques | plaques in | in 2 | in 2 | in 2 | in 2 | in 2 | plaques in 2 |
| Virus | in 2 dishes | 2 dishes | dishes | dishes | dishes | dishes | dishes | dishes |
| 10⁻³ | C* | — | 1 | — | — | — | — | 3 |
| 10⁻⁴ | 80 | — | — | — | — | — | — | 1 |
| 10⁻⁵ | 8 | — | — | — | — | — | — | — |
| 10⁻⁶ | 1 | — | — | — | — | — | — | — |
| 10⁻⁷ | — | — | — | — | — | — | — | — |

C* = confluent plaques

Compounds Tested at the Same Molar Concentration of a 12.5 γ/ml Xenaldial Solution

2) Virus: Rhinovirus H Strain, Cell Line: Ohio-HeLa

Results:

| | | | DV1 | DV2 | DV3 | DV4 | DV5 | |
|---|---|---|---|---|---|---|---|---|
| Dilution | Non-treated | Xenaldial | n^{o} of | n^{o} of | n^{o} of | n^{o} of | n^{o} of | Xenalamine |
| of | control n^{o} | n^{o} of | plaques | plaques | plaques | plaques | plaques | n^{o} of |
| inoculated | of plaques | plaques in | in 2 | in 2 | in 2 | in 2 | in 2 | plaques in 2 |
| Virus | in 2 dishes | 2 dishes | dishes | dishes | dishes | dishes | dishes | dishes |
| 10⁻³ | C* | — | 1 | — | — | 1 | — | 2 |
| 10⁻⁴ | 88 | — | — | — | — | 1 | — | 1 |
| 10⁻⁵ | 9 | — | — | — | — | — | — | 1 |
| 10⁻⁶ | 2 | — | — | — | — | — | — | — |
| 1O⁻⁷ | — | — | — | — | — | — | — | — |

C* = confluent plaques

Compounds Tested at the Same Molar Concentration of a 12.5 γ/ml Xenaldial Solution

3) Virus: Rhinovirus M Strain, Cell Line: MKC-5

Results:

| | | | DV1 | DV2 | DV3 | DV4 | DV5 | |
|---|---|---|---|---|---|---|---|---|
| Dilution | Non-treated | Xenaldial | n^{o} of | n^{o} of | n^{o} of | n^{o} of | n^{o} of | Xenalamine |
| of | control n^{o} | n^{o} of | plaques | plaques | plaques | plaques | plaques | n^{o} of |
| inoculated | of plaques | plaques in | in 2 | in 2 | in 2 | in 2 | in 2 | plaques in 2 |
| Virus | in 2 dishes | 2 dishes | dishes | dishes | dishes | dishes | dishes | dishes |
| 10⁻³ | C* | — | 1 | — | — | 1 | — | 4 |
| 10⁻⁴ | 84 | — | 1 | — | — | 1 | — | 2 |
| 10⁻⁵ | 6 | — | — | — | — | — | — | 1 |
| 10⁻⁶ | 2 | — | — | — | — | — | — | — |
| 10⁻⁷ | — | — | — | — | — | — | — | — |

C* = confluent plaques

Compounds Tested at the Same Molar Concentration of a 12.5 γ/ml Xenaldial Solution

### EXAMPLE 2

### In-vitro Activity of p-(α-ethoxy-β-keto-β-2-naphthyl-ethylamino-benzoic acid (DV2)

The activity of D2 was evaluated at progressive dilutions in HeLa cells infected with HR14 Rhinovirus and grown in Dulbecco's medium added with 2% fetal calf serum.

D2 was dissolved in dimethylsulfoxide (DMSO) at 100 mg/ml and diluted to 3 mg/ml with Dulbecco's medium.

All the experiments were carried out in duplicate.

The maximum non toxic dose (MNTD) and the minimum inhibitory concentration (MIC) determined by means of electronic microscope were 51 μg/ml and 0.006 μg/ml respectively. The protection index calculated as the ratio MNTD/MIC is 8.5, which is much higher than that of other known anti-Rhinovirus compounds such as Disoxaril. These results show that DV2 is active at low dose against HR14 Rhinovirus and is devoid of toxicity against HeLa cells.

### EXAMPLE 3

### Pharmaceutical Compositions

A chewable effervescent tablet having the following composition was prepared:

| | |
|---|---|
| active ingredient (compounds DV1-DV5, | mg 35 |
| xenalamine or xenaldial) = | |
| corn starch = | mg 8 |
| tribasic citrate = | mg 40 |
| magnesium stearate = | mg 2 |

Spray/Aerosol

Spray: 10 ml container (about 100 doses) with single dose dispenser (extractable volume 0.1 ml/dose). Composition (w/w):

| | |
|---|---|
| active ingredient = | 15% |
| thymol = | 0.84% |
| tannic acid = | 1.68% |
| alcohol = | 45% |
| purified water to 100, about = | 37.08% |

Aerosol: nebulizer-fittable 1 ml monodose vials.
Same composition as the spray.

### REFERENCES

B. Lotti et al.: in Boll. Soc. It. Biol. Sperim.: 38, 1308 (1962)
P. Altucci et al.: in Giorn. Microbiol.: 9, 186 (1961)
P. Altucci et al.: in Boll. Sierot. Mil.: 40, 626 (1961)
F. Coraggio et al.: in Boll. Soc. It. Biol. Sperim.: 32, 1432 (1960)
F. Coraggio et al.: in Atti II Simp. Internaz. Chemiot.: 268
G. Tarro et al.: in Boll. Soc. It. Biol. Sperim.: 1353 (1962)
G. Tarro et al.: in Boll. Soc. It. Biol. Sperim.: 1355 (1962)
G. Tarro et al.: in Boll. Soc. It. Biol. Sperim.: 1356 (1962)
P. F. Lin, B. Robinson, Y. F. Gong, K. Ricardi, Q. Guo, C Deminie, R. Rose, T. Wang, N. Meanwell, Z. Yang, H. Wang, T. Zang and R. Colonno: “Identification and Characterization of a Novel Inhibitor of HIV-1 Entry-I: Virology and Resistance”; 9^{th} Conference on Retroviruses and Opportunistic Infectious, Feb. 24-28, 2002.
P. F. Lin, K. Guo, R. Fridell, H. T. Ho, G. Yamanaka and R. Colonno: “Identification and Characterization of a Novel Inhibitorn of HIV-1 Entry-I: Mechanism of Action”; 9^{th} Conference on Retroviruses and Opportunistic Infectious, Feb. 24-28, 2002.
J. Reynes, R. Rouzier, T. Kanouni, V. Baillat, B. Baroudy, A Keung, C. Hogan, M. Markowitz, M. Laughlin: “SCH-C: Safety and Antiviral Effects of a CCR5 Receptor Antagonist in HIV-1 Infected Subjects”; 9^{th} Conference on Retroviruses and Opportunistic Infectious, Feb. 24-28, 2002.
J. Ripley, L. Wojcik, S. Xu, J. Strizki: “Genotypic and Phenotypic Analysis of in-Vitro Generated HIV-1 Escape Isolates to the CCR5 Antagonist SCH-C”; 9^{th} Conference on Retroviruses and Opportunistic Infectious, Feb. 24-28, 2002.
S. Xu, L. Wojcik, J. Strizki: “Antagonism of the CCR5 Receptor by SCH-C Leads to Eleveted beta-Chemochine Kevels and Receptor Expression in Chronically treated PBMC Cultures”; 9^{th} Conference on Retroviruses and Opportunistic Infectious, Feb. 24-28, 2002.

1. A method for prevention or treatment of Rhinovirus infections comprising the step of administering an effective amount of a compound selected from the group consisting of:
a) p-(α-ethoxy-p-phenyl-phenacylamino)-benzoic acid (xenalamine): 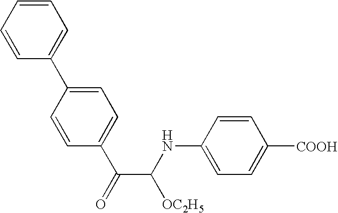
b) 4,4′-bis-biphenylglyoxal-hydrate (xenaldial): 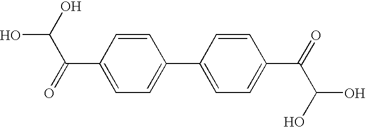
c) p-(α-ethoxy-β-keto-β-1-naphthyl)-ethylamino-benzoic acid (DV1): 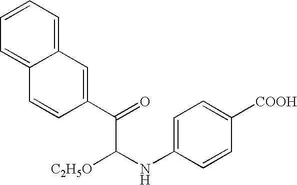
d) p-(α-ethoxy-β-keto-β-2-naphthyl)-ethylamino-benzoic acid (DV2): 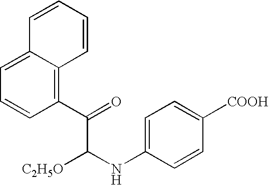
e) p-(α-ethoxy-β-keto-β-2-cumaroyl)-ethylamino-benzoic acid (DV3): 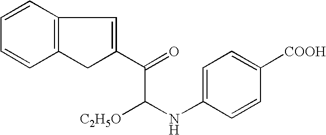
f) p-(α-ethoxy-β-keto-β-1-phenanthryl)-ethylamino-benzoic acid 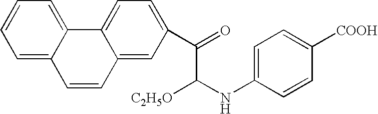
g) p-(α-ethoxy-β-keto-β-2-phenanthryl)-ethylamino-benzoic acid (DV5):

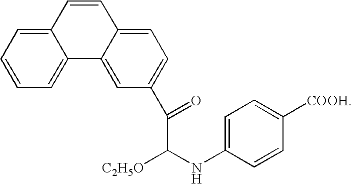

2. The method of claim 1 for the prevention or the treatment of said infections of the upper, middle and lower respiratory tract.

3. The method of claim 1, for the prevention or treatment of colds.

4-6. (canceled)

7. A pharmaceutical composition for inhalatory administration comprising a compound selected from the group consisting of:
a) p-(α-ethoxy-p-phenyl-phenacylamino)-benzoic acid (xenalamine): 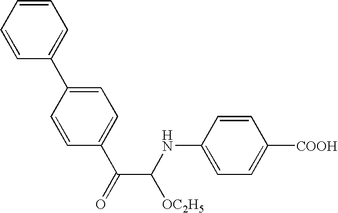
b) 4,4′-bis-biphenylglyoxal-hydrate (xenaldial): 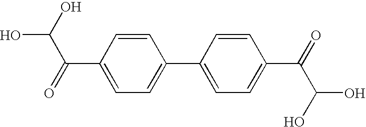
c) p-(α-ethoxy-β-keto-β-1-naphthyl)-ethylamino-benzoic acid (DV1): 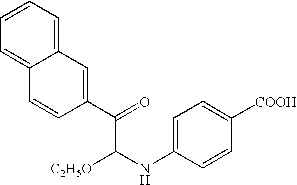
d) p-(α-ethoxy-β-keto-β-2-naphthyl)-ethylamino-benzoic acid (DV2): 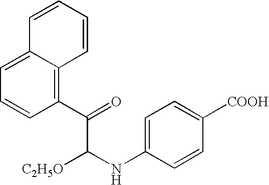
e) p-(α-ethoxy-β-keto-β-2-cumaroyl)-ethylamino-benzoic acid (DV3): 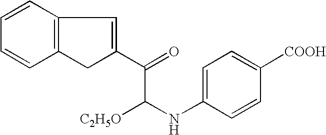
f) p-(α-ethoxy-β-keto-β-1-phenanthryl)-ethylamino-benzoic acid (DV4): 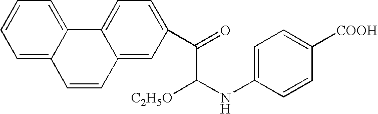
g) p-(α-ethoxy-β-keto-β-2-phenanthryl)-ethylamino-benzoic acid (DV5):

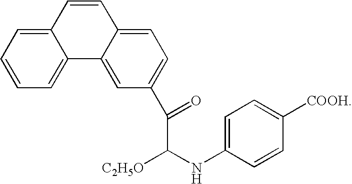

8. The composition as claimed in claim 7, in the form of at least one of a solution, suspension, dispersion, powder and granulate.

9. The composition of claim 7, in the form of a spray.

10. The composition of claim 8, in the form of a spray

11. The composition of claim 7, in the form of an aerosol.

12. The composition of claim 8, in the form of an aerosol.
